(19) United States
(12) Patent Application Publication
(10) Pub. No.: US 2014/0260561 A1
(43) Pub. Date: Sep. 18, 2014

(54) SOLVENT-FREE METHOD FOR MEASURING HYDROCARBONS IN WATER

(71) Applicants: Dale Brost, Coarsegold, CA (US); Amy Foster, Clovis, CA (US)

(72) Inventors: Dale Brost, Coarsegold, CA (US); Amy Foster, Clovis, CA (US)

(21) Appl. No.: 14/213,227

(22) Filed: Mar. 14, 2014

Related U.S. Application Data
(60) Provisional application No. 61/799,725, filed on Mar. 15, 2013.

(51) Int. Cl. G01N 33/28; G01N 21/64

(52) U.S. Cl. CPC G01N 33/28; G01N 21/64
USPC 73/61.48

(57) ABSTRACT

The present application is directed to methods for measuring hydrocarbons in a water sample. The water sample may be diluted by a dilution factor to reduce the salinity level. An amount of surfactant determined by the dilution factor and the critical micelle concentration of the surfactant may be added to the water sample. The hydrocarbon concentration may be determined by fluorescence measurement.

### CROSS-REFERENCE TO RELATED APPLICATIONS

The present application claims priority to provisional U.S. Patent Application Ser. No. 61/799,725, filed on Mar. 15, 2013, titled “No-Solvent Method for Measuring Hydrocarbons in Water and Soil with Fluorescence Detection Instruments and Means for Multi-Axis Optical Measurements of Fluid Streams with Sonic Cleaning and Homogenization,” which is hereby incorporated by reference in its entirety.

### FIELD OF THE INVENTION

The present application is directed generally to analytical measurement techniques, and more specifically to methods for determining the hydrocarbon concentration of a water sample using fluorescence.

### BACKGROUND

In the United States alone, the number of producing natural gas wells in service in 2011 is estimated to be 504,000 and the number of producing oil wells is estimated to be 536,000. Due to the continued and expanding use of horizontal drilling combined with hydraulic fracturing, the number of producing oil and gas wells is expected to significantly increase. These drilling techniques allow oil and gas to be extracted from formerly unproductive unconventional formations such as shale deposits or plays and coalbed methane deposits. Each of these wells has the potential to generate large volumes of produced water.

The U.S. Environmental Protection Agency has defined produced water as “the water (brine) brought up from the hydrocarbon bearing formation strata during the extraction of oil and gas, and can include formation water, injection water, and any chemicals added downhole or during the oil/water separation process.” Produced water may contain dissolved inorganic salts and organic compounds, dispersed oil droplets, dissolved gases, bacteria, and dispersed solid particles. Accordingly, environmental regulations are regulating the disposal of these fluids. Proper analysis of the constituents present in produced water is essential to efficiently and cost effectively treat the water.

Most oil-in-water analysis methods for produced water require the oil to be extracted into an organic solvent prior to measurement. Many of the organic solvents used for extraction are either extremely flammable, hazardous to human health or both. The chlorinated hydrocarbons are very expensive and must be either recycled or disposed of as hazardous waste. Volatile hydrocarbon solvents such as pentane and hexane are extremely flammable and present a serious fire and explosion risk.

### SUMMARY

Various embodiments of the methodology presented here make it possible to perform oil-in-water analyses by making measurements directly on the produced water sample. No organic solvents are required. Various embodiments are based upon the addition of a detergent surfactant to a produced water sample. The surfactant may convert the dispersed oil in the water sample into an optically clear microemulsion that is ideal for direct fluorescence measurements using a TD-500D Oil-in-Water Analyzer (Turner Designs Hydrocarbon Instruments, Inc., Fresno, Calif.). The surfactant is safe to handle with a minimum of personal protective equipment and is only slightly flammable even under a direct flame. The U.S. Department of Transportation does not consider it to be a hazardous material. It can be shipped without hazardous identification labels and can be carried on commercial airlines and helicopters without declaration.

Two samples (“Background” and “OIW”) are collected to perform an analysis. The Background sample is untreated produced water. It is filtered into a measurement cuvette through a 0.2 micron syringe filter. The filter removes suspended solids and dispersed oil. Only water-soluble substances pass through the filter into the cuvette. The OIW sample is collected into a bottle containing surfactant. The sample is then heated to the cloud point of the surfactant and allowed to cool until the cloudiness disappears. This converts the dispersed oil into a stable microemulsion. The dispersed oil is located inside micelles that are small enough to pass through a 0.2 micron filter. The converted OIW sample is then filtered into a cuvette. The TD-500D readings for the Background and OIW cuvettes are then recorded. The dispersed oil concentration is the calculated difference between the OIW and Background readings. The Background reading itself provides additional information. Since it is proportional to the concentration of fluorescent water-soluble organics in the produced water sample, the Background reading can be used to track changes in the concentrations of these substances.

The TD-500D has two measurement channels, A and B. Channel A makes fluorescence measurements using ultraviolet light and is used when the highest sensitivity is required. Channel B uses visible light for reduced sensitivity and extended dynamic range. With the solvent-free method, channel A detects most crude oils at dispersed oil concentrations less than 1 ppm. The linear range is from 0 to at least 100 ppm. The dynamic range can typically be extended to 750 ppm or greater with a non-linear calibration function. When the instrument is set to channel B, various embodiments of the solvent-free method can measure dispersed oil concentrations up to 10,000 ppm, eliminating the need to dilute highly concentrated oil-in-water samples.

The present application is directed to methods for measuring hydrocarbons in a water sample. An exemplary method may comprise determining a salinity value of the water sample. A dilution factor may then be determined to reduce the salinity value of the water sample to less than 150,000 parts per million (ppm) of total dissolved solids (TDS). The water sample may be diluted by the dilution factor using essentially hydrocarbon-free deionized water. An amount of surfactant may then be added to the diluted water sample. The amount of surfactant added may be equal to about the critical micelle concentration of the surfactant multiplied by the dilution factor. The hydrocarbon concentration of the diluted surfactant treated water sample may then be measured.

According to additional exemplary embodiments, the present application may be directed to further methods for measuring hydrocarbons in a water sample. While some embodiments may be carried out at least in part manually, other embodiments may be performed utilizing one or more non-transitory computer readable media having computer-executable instructions for performing a method by running a software program on a computer, the computer operating under an operating system, the method comprising instructions from the software program for measuring hydrocarbons in a water sample. The instructions may comprise determining a salinity value of a first water sample. A dilution factor may then be determined to reduce the salinity value of the first water sample to less than about 150,000 ppm of total dissolved solids. The first water sample may then be diluted by the dilution factor using essentially hydrocarbon-free deionized water. An amount of surfactant may be added to the diluted first water sample. The amount of surfactant added may be equal to about the critical micelle concentration of the surfactant multiplied by the dilution factor. A second water sample may be prepared by filtering a portion of the water sample, then diluting the filtered second water sample by the dilution factor using essentially hydrocarbon-free deionized water. The fluorescence of the diluted surfactant treated first water sample and the diluted second water sample may be measured. The hydrocarbon concentration of the water sample may be determined based on a difference between the fluorescence measurements of the diluted surfactant treated first water sample and the diluted second water sample.

### BRIEF DESCRIPTION OF THE DRAWINGS

FIG. 1 is a schematic diagram of the fluorescence process.

FIG. 2 is a perspective view of a fluorometer.

FIG. 3 is a schematic diagram of a surfactant molecule.

FIG. 4 is a schematic diagram of the solubilization of dispersed oil.

FIG. 5 is a schematic diagram of aliphatic alcohol ethoxylate surfactants.

FIG. 6 is a graph of oil concentration plotted with fluorometer readings for the linear range determination and 2^{nd} order polynomial correlation for Tyra East oil according to various embodiments.

FIG. 7 is a graph of relative fluorescence plotted with dispersed oil concentration to demonstrate the effect of heating surfactant treated water samples according to various embodiments.

FIG. 8 is a graph of measured oil concentration plotted with actual oil concentration according to various embodiments.

FIG. 9 is a graph of relative fluorescence plotted with oil concentration for Oseberg C oil according to various embodiments.

FIG. 10 is a schematic facility diagram.

FIG. 11 is a flowchart of an exemplary method for measuring hydrocarbons in a water sample according to various embodiments.

FIG. 12 is a flowchart of an exemplary method for measuring hydrocarbons in a water sample according to various embodiments.

### DETAILED DESCRIPTION

Aromatic fractions of dispersed oil and water-soluble organics (WSO) found in produced water can be stimulated to emit fluorescent light. The process is illustrated in FIG. 1. Excitation light is directed to a sample at a wavelength, λ_{EX}. The aromatic molecules in the sample absorb the excitation light and jump from their normal energy level (E₀, ground state) to an excited energy state, E₂. The excited molecules then lose some of their absorbed energy by a variety of mechanisms (relaxation) and go to a lower energy state, E₁. The molecules then drop back down to E₀ by emitting a photon of fluorescent light at a wavelength λ_{EM}. The energy emitted by fluorescence (E₁−E₀) is lower than the energy gained by absorption (E₂−E₀). Since light energy is inversely proportional to wavelength, the wavelength of the fluorescent light, λ_{EM}, is always longer than the wavelength of the excitation light, λ_{EX}. The intensity of the fluorescence emission, I_{F}, is proportional to the concentration of the fluorescent molecules in the sample.

Fluorescence is an extremely sensitive analytical technique, capable of monitoring most oils in produced water at concentrations less than 1 mg/L. Commercial fluorometers are available that are designed to monitor the oil content of grab-samples. Others are designed to monitor oil on-line in a flowing produced water stream. FIG. 2 shows the TD-500D, a dual-range, hand-held fluorometer for the analysis of grab-samples.

The TD-500D is a battery-powered, solid-state fluorometer, with two analysis channels, A and B, operating at different wavelengths. Channel A makes fluorescence measurements at short wavelengths, with fluorescence excitation in the ultraviolet portion of the spectrum. Channel B operates at longer wavelengths with fluorescence excitation in the visible range. The instrument stores calibration values for both optical channels. Channel A is typically used to measure oil concentrations in the range normally encountered at the effluent of a water-purification system. Channel B is typically used for highly fluorescent oils or for high-concentration samples where reduced sensitivity is required. For added flexibility, two sample cuvettes are available, with internal diameters of 2 mm and 8 mm, respectively.

The instrument is calibrated with a blank and a crude oil standard. If operating in a range where fluorescence is a linear function of oil concentration (normal overboard discharge concentrations), the TD-500D can be calibrated to read out directly in ppm. If operating over an extended concentration range, where the fluorescence versus concentration relationship is non-linear, the TD-500D readings can be mathematically converted to oil concentration using an equation or look-up table.

Various commercial fluoroscopy methods to determine the hydrocarbon concentration in a water sample include a step that uses a non-fluorescent organic solvent to extract the oil. While the solvent extract method may be effective to extract all of the oil from the water sample for analysis, it also has numerous disadvantages. Organic solvents are generally considered to be hazardous substances. Many are extremely flammable, toxic or both. All must be handled with extreme care and disposed of in an environmentally responsible manner. Other problems can be associated with the 10× concentration effect arising from the extraction process. Although the concentration enhancement is sometimes beneficial when monitoring low concentrations of weakly fluorescent oils (e.g., refined hydrocarbons, condensates, etc.), it can result in a limited measurement range for highly fluorescent oils. Therefore, the solvent extract method has limited commercial viability.

Solubilizing Dispersed Oil with Surfactants

The word surfactant is an acronym for surface active agent. As illustrated in FIG. 3, a surfactant molecule is bipolar in nature, composed of a hydrophilic (strong affinity for water) end and a lypophilic (strong affinity for oil) end. The bipolar structure of surfactant molecules allows them to modify the properties of oil/water mixtures. Some types of surfactants can stabilize water-in-oil emulsions. Others stabilize oil-in-water emulsions. Still others act as detergents and are commonly used to remove oil from solid surfaces (metal, china, ceramics, skin, hair, etc.). The general suitability of a surfactant for a particular purpose can be determined by its hydrophilic-lypophilic balance (HLB):

HLB less than 10: Stabilize water-in-oil emulsions

HLB greater than 10: Stabilize oil-in-water emulsions

HLB 12 to 15: Detergents

As shown in FIG. 4, when a detergent surfactant is added to a produced water stream containing dispersed oil droplets, the lypophilic end adsorbs into the oil droplets, leaving the hydrophilic end in contact with the surrounding water molecules. This process reduces the interfacial tension between the oil and the water, which, with the addition of energy (agitation, shear, heat, etc.) breaks up the oil droplets into much smaller particles. If the surfactant is present above its critical micelle concentration, CMC, the oil becomes incorporated into the lypophilic cores of micelles. Micelles are extremely tiny structures with typical diameters of 0.003-0.2 μm. They are physically stable and spontaneously disperse themselves uniformly throughout a water sample. The conversion of a heterogeneous mixture of dispersed oil in water to a homogeneous, stable, micellar dispersion (microemulsion) is called “solubilization.” Micelles are so small that microemulsions do not scatter visible light. As a result, the microemulsions are optically clear even at very high oil concentrations.

The critical micelle concentration is defined as the concentration of surfactants above which micelles form and all additional surfactants added to the system go to micelles. The value of the CMC for a given surfactant in a given medium will depend on temperature, pressure, and the presence and concentration of other surface active substances in the system.

Many types of detergent surfactants are available. The aliphatic alcohol ethoxylates (AAE) have been found to be effective for solubilizing crude oil in produced water. As illustrated by the general chemical structures shown in FIG. 5, the lypophilic ends of these surfactants contain various types of hydrocarbons. The hydrophilic ends are polymers of ethylene oxide. A simplified schematic representation is shown to the right of each chemical formula. In addition, alkyl phenol ethoxylates have been found to be effective for solubilizing crude oil.

AAE surfactants have cloud points. The cloud point is the maximum temperature at which the surfactant is completely soluble in water. When the temperature of an aqueous solution of AAE surfactant reaches its cloud point, most of the surfactant comes out of solution as a separate, surfactant-rich phase, and the once clear solution becomes a cloudy mixture.

The surfactant-rich phase may contain 50-80% surfactant and acts as an effective solvent for crude oil. This solvent-like behavior helps the surfactant capture dispersed oil that is embedded in suspended solids and stuck to the walls of the sample bottle. The cloud point phase-behavior is reversible. When the temperature of the mixture drops back below the cloud point, the surfactant-rich phase disappears, the optically-clear microemulsion structure is re-established (i.e., the cloudiness abates), and the sample is once again suitable for fluorescence measurements.

The oil solubilization power and cloud point temperature of an AAE surfactant depends upon its chemical structure and the salinity and hardness of the water in which it is dissolved. Various embodiments comprise an AAE surfactant that efficiently converts all types of crude oils and condensates into clear microemulsions that are ideal for fluorescence analysis. The surfactant blend works with water samples containing a wide range of salinity and hardness. The effective cloud point temperature is slightly above ambient, making it easy to raise the sample above the cloud point for maximum solubilization and cool it again for optimum fluorescence measurement. It is also non-flammable, safe to handle and has minimal fluorescence at the analysis wavelengths of the TD-500D Oil-in-Water Analyzer.

Solvent-Free Oil-In-Water Analysis Method

Overview

Various embodiments were designed to measure the dispersed oil concentration of produced water from the outlet of the first oil/water separator to the point of final discharge. For a typical North Sea crude oil, the dynamic range of the analysis is 0-10,000 ppm (without dilution). The method was also designed to be independent of salinity and hardness over a broad range (0-120,000 ppm TDS). In addition to measuring dispersed oil concentration, the method also provides a separate measurement of the non-oil, fluorescent background of the water sample, which can be used to monitor changes in the concentration of fluorescent water-soluble organics.

Synthetic Produced Water Samples

Synthetic produced water samples were used to develop and characterize the method. Synthetic produced water samples were prepared by adding known amounts of crude oil and inorganic salts to filtered produced water from the Kern River Field, a mature, steam-flood project in southern California. Kern River produced water was selected because it contains less than 1,000 ppm TDS. This made it possible to add different amounts of an inorganic salt mixture to create brines with a wide range of salinity and hardness. Kern River produced water was also ideal for this study because it contains a substantial concentration of highly-fluorescent water-soluble organics (WSO). The synthetic brines used in this study are shown in Table 1.

TABLE 1
Synthetic Brine Compositions¹

| Component | Brine 1 | Brine 2 | Brine 3 |
|---|---|---|---|
| Na⁺ | 10,228 | 20,455 | 40,911 |
| Ca²⁺ | 1,425 | 2,849 | 5,698 |
| Mg²⁺ | 293 | 587 | 1,174 |
| Cl⁻ | 19,149 | 38,298 | 76,596 |
| TDS | 31,095 | 62,189 | 124,379 |
| WSO² | 16 | 16 | 16 |

¹Concentration values are expressed in units of mg/L
²Water-soluble organics from Kern River produced water, concentrations are expressed as mg/L as determined by EPA Method 1664.

Dispersed oil and suspended solids were quantitatively removed by passing the produced water through two syringe filters arranged in series. Each filter contained a glass fiber pre-filter and a 0.2 micron cellulose acetate membrane. Laboratory studies showed that this filter arrangement removed more than 99.5% of dispersed North Sea crude oil after shearing a 100 ppm oil-in-water sample in a high-shear mixer at 24,000 revolutions per minute.

Samples of known dispersed oil content were created by adding measured amounts of various test oils to Brine 1, Brine 2 or Brine 3. The oils included in this study included a variety of crude oils from the North Sea, heavy oil from California and a condensate from the Gulf of Mexico.

TD-500D Configuration

The TD-500D was configured for this analysis method by setting the instrument to channel A and installing a special 8 mm cuvette adaptor (with a 0.06″ optical aperture, Part #102680) in the sample compartment. All fluorescence measurements were made on water samples in 8 mm cuvettes.

Calibration

A 100 ppm standard was prepared by dissolving 10 μL of crude oil in 3 mL of surfactant. Distilled water was added until the total volume was 100 mL. The resulting mixture was shaken until all the surfactant dissolves. The mixture was then heated to the cloud point of the surfactant and cooled to room temperature. The 100 ppm standard was filtered into an 8 mm cuvette using a 0.2 micron cellulose acetate syringe filter. A blank was prepared by adding 3 mL of surfactant to a graduated bottle, and then filling the bottle to the 100 mL mark with distilled water. After the surfactant dissolved, the blank was placed in another 8 mm cuvette without filtration. The TD-500D was then calibrated as described in the user's manual.

After the calibration was completed, the calibration diagnostic button, <DIAG>, was pressed and the values for the % full-scale for the blank and standard were recorded (% FS-BLK and % FS-STD). These values represent the fluorescence intensity emitted by the blank and standard, expressed as a percentage of the full-scale measurement capacity of the TD-500D. The % FS-BLK provides a check on the fluorescence of the blank (3% surfactant in distilled water). It should be fairly low, with a typical value of <1%. The % FS-STD is a measure of the fluorescence of the 100 ppm standard after the % FS-BLK is subtracted. Its value should be ≧3% for a 100 ppm standard, in order to achieve adequate precision with dispersed oil concentrations in the 0-30 ppm range.

If the % FS-STD value is <3% for a 100 ppm standard, it is necessary to increase the sensitivity of the TD-500D by using an 8 mm cuvette adaptor with a larger optical aperture (Part #102852). The larger aperture increases the amount of fluorescent light that is sensed by the light detector. The result is a larger % FS-BLK (typical values of 2-4%) and % FS-STD. The minimum effective % FS-STD required to precisely measure dispersed oil concentration in the 0-30 ppm range is the same as mentioned above for the smaller aperture (3% for a 100 ppm standard).

Linear Range Determination

With various embodiments, most types of oil give TD-500D readings (Channel A) that are linear with oil concentration to at least 100 ppm. The exceptions are some heavy crudes (<13° API), which have lower linearity limits (typically ˜80 ppm). Some oils have linearity limits greater than 100 ppm. At oil concentrations above the linearity limit, a TD-500D reading is a non-linear function (2nd-order polynomial) of oil concentration. Using a non-linear correlation function makes it possible to measure oil concentrations up to 750 ppm or greater with a typical North Sea crude oil without dilution of the sample.

The linearity limit and the 2nd-order polynomial correlation function may be readily determined for any type of oil through the use of control samples. The following discussion describes how to do this for oil concentrations up to 1000 ppm.

A 1000 ppm stock control sample was made by dissolving 100 μL of crude oil in 3 mL of surfactant, adding distilled water to a total volume of 100 mL, followed by heating to the cloud point and cooling to room temperature. Three control samples were created with concentrations of 250 ppm, 500 ppm and 750 ppm by diluting the 1000 ppm stock with a solution of 3% surfactant in distilled water. Each control sample was then filtered into a cuvette through a separate 0.2 micron syringe filter. Separate filters are used to avoid contaminating one control sample with another.

TD-500D readings were then taken for the 100 ppm standard and for each control sample (250 ppm, 500 ppm, 750 ppm and 1000 ppm). The results were plotted and fit with a 2nd-order polynomial as shown in FIG. 6 for Tyra East crude oil from the Danish North Sea. The form of the 2nd-order polynomial is:

Oil Concentration,ppm=ax²+bx Equation 1

where:

x represents the TD-500D reading,

a is the regression coefficient of x², and

b is regression coefficient of x.

The linearity limit can be estimated graphically as illustrated in FIG. 6, or calculated directly using Equation 2.

Linearity Limit,ppm=(1−b)/a Equation 2

where: a is the coefficient of x², and

b is the coefficient of x.

For the Tyra East example, the linearity limit calculated by Equation 2 is 139 ppm.

Sample Analysis

Two samples (“Background” and “OIW”) were collected to perform an analysis. The Background sample was untreated produced water. The Background sample was filtered into a measurement cuvette through a 0.2 micron syringe filter. The filter removed suspended solids and dispersed oil. Only water-soluble substances pass through the filter into the cuvette. The OIW sample was collected into a bottle containing surfactant. The OIW sample was then heated to the cloud point of the surfactant and allowed to cool until the cloudiness disappears. The heating and subsequent cooling of the OIW sample converted the dispersed oil into a stable microemulsion. The dispersed oil was located inside micelles that are small enough to pass through a 0.2 micron filter. The converted OIW sample was then filtered into a cuvette. The TD-500D readings for the Background and OIW cuvettes were then recorded. If the OIW reading and the Background readings are both less than or equal to the linearity limit, the dispersed oil concentration is the calculated difference between the OIW and Background readings, as shown in Equation 3.

Oil Concentration,ppm=OIW Reading−Background Reading Equation 3

However, if the TD-500D reading is greater than the linearity limit, the actual oil concentration should be computed by Equation 1, using the result of Equation 3 (OIW Reading−Background Reading) for the value of x.

The Background reading itself provides additional information. Since it is proportional to the concentration of fluorescent water-soluble organics in the produced water sample, the Background reading can be used to trend these substances.

Effect of Heating the Sample to the Cloud Point

Heating the surfactant-treated sample to the cloud point may be beneficial because of the solvent-like properties of the surfactant-rich phase. To illustrate this, three sets of samples were created by adding measured amounts of Troll C oil to Brine 1 (Table 1). Each set contained three samples with oil concentrations of 0 ppm, 25 ppm and 50 ppm, respectively. Each set was analyzed as described above, but the standards and samples were heated to different temperatures. The first set was left at room temperature, the second set was heated to the cloud point, and the third set was heated to boiling. The 50 ppm sample from each set was used to calibrate the TD-500D. After calibration, the dispersed oil concentrations of all three samples were measured. The effect of heating the samples to different temperatures is shown in FIG. 7.

Heating the samples to the cloud point significantly increased the amount of fluorescence emitted by each dispersed oil concentration. This proves that heating to the cloud point effectively increases the surfactant's effectiveness. Increasing the temperature to boiling resulted in an additional increase, but the amount of the increase was very small. This shows that it is not important to carefully control the temperature to achieve consistent results. Any temperature at or above the cloud point will give essentially the same fluorescence yield.

Effect of Salinity and Hardness

To determine the effect of salinity and hardness, the TD-500D was calibrated with a standard containing 100 ppm of Troll C oil in Brine 1. Three samples containing 50 ppm of Troll C oil were prepared, each with a different salinity and hardness (Brine 1, Brine 2 and Brine 3). The analysis results for the samples are shown in Table 2.

TABLE 2
Effect of Salinity and Hardness

| | | Actual Dispersed | Measured Dispersed |
|---|---|---|---|
| | | Oil Concentration, | Oil Concentration, |
| Sample | Brine | ppm | ppm |
| Blank | Distilled Water | 0 | — |
| Standard | Brine 1 | 100 | — |
| 1 | Brine 1 | 50 | 50.3 |
| 2 | Brine 2 | 50 | 50.0 |
| 3 | Brine 3 | 50 | 49.8 |

Even though the instrument was calibrated with Brine 1, the measured dispersed oil concentrations in all 3 brines are in excellent agreement with the actual values. This shows that the method is not affected by gross changes in salinity and hardness.

Precision Estimate

To estimate the precision of the method, 10 samples, each with an oil concentration of 30±1 ppm, were created and analyzed. The TD-500D was operated on Channel A and calibrated as described above using a 100 ppm standard of oil from the Dan Field in the Danish North Sea. The % FS-STD was 11.2, indicating that the standard was fluorescent enough for good measurements at 30 ppm. The samples were prepared by adding 3 μL of Dan oil and 10 mg of powdered iron oxide to 100 mL of Brine 1. To simulate worst-case volumetric precision, the standard and samples were prepared using the graduations on the sample bottles (180 mL glass prescription bottles). High-precision volumetric glassware was not used. TD-500D readings and analysis results are shown in Table 3. The absolute uncertainties of the standard and sample concentrations are also given.

TABLE 3
Precision Estimate

| | | |
|---|---|---|
| OIW | Background | Dispersed Oil, ppm |

| Sample | Reading | Reading | Actual | Measured |
|---|---|---|---|---|
| 1 | 51.3 | 20.8 | 30 ± 1¹ | 30.5 |
| 2 | 51.3 | 21.0 | 30 ± 1 | 30.3 |
| 3 | 54.6 | 20.6 | 30 ± 1 | 34.0 |
| 4 | 51.3 | 21.0 | 30 ± 1 | 30.3 |
| 5 | 50.4 | 20.5 | 30 ± 1 | 29.9 |
| 6 | 51.5 | 20.8 | 30 ± 1 | 30.7 |
| 7 | 50.4 | 20.8 | 30 ± 1 | 29.6 |
| 8 | 51.5 | 20.0 | 30 ± 1 | 31.5 |
| 9 | 52.2 | 21.1 | 30 ± 1 | 31.1 |
| 10 | 54.0 | 20.8 | 30 ± 1 | 33.2 |

| | |
|---|---|
| Mean, ppm | 31.1 |
| σ, ppm | 1.4 |
| Relative Uncertainty, % of Mean | 4.1 |
| Relative Uncertainty, % of Range | 2.9 |
| (Range = 100 ppm) | |

¹Absolute uncertainty, estimated by normal propagation of random error.

The average disbursed oil concentration was 31.1 ppm. The standard deviation of the dispersed oil analysis results was 1.4 ppm, which is just slightly greater than the estimated precision of the samples. The concentration values were equally distributed about the mean of 31.1 ppm, indicating that the error was random and not systematic. The relative uncertainty was 4.1% at the 30 ppm oil concentration level.

To determine overall accuracy, samples containing a wide range of oil types were prepared in Brine 1 at dispersed oil concentrations from 2 to 80 ppm and analyzed. With the exception of the condensate, all the oils were measured on Channel A, using 8 mm cuvettes and a small-aperture sample adaptor. The condensate samples were much less fluorescent and required the large-aperture sample adaptor to achieve optimum sensitivity. The TD-500D was calibrated with the 100 ppm sample and a distilled-water blank. Samples and standards were prepared as described above, using the graduations on the sample bottles. Analysis results are reported in Tables 4 and 5. The absolute value of the error (|Measured−Actual|) for each sample is given in the last column. A composite accuracy plot, (Measured Concentration vs. Actual Concentration), including all the oils, is shown in FIG. 8.

TABLE 4
Accuracy Determination, Part 1

| | Concentration, ppm | | \|Error\|, |
|---|---|---|---|
| Oil Source | Actual | Measured | ppm |
| Brage, North Sea | 2 | 4 | 2 |
| | 6 | 8 | 2 |
| | 20 | 21 | 1 |
| | 40 | 39 | 1 |
| | 60 | 62 | 2 |
| | 80 | 82 | 2 |
| Average \|Error\|, % of Range | | | 1.7 |
| Grane, North Sea | 2 | 4 | 2 |
| | 6 | 7 | 1 |
| | 20 | 20 | 0 |
| | 40 | 42 | 2 |
| | 60 | 59 | 1 |
| | 80 | 77 | 3 |
| Average \|Error\| [symbol] % of Range | | | 1.5 |
| Oseberg C, North Sea | 2 | 5 | 3 |
| | 6 | 7 | 1 |
| | 20 | 20 | 0 |
| | 40 | 40 | 0 |
| | 60 | 59 | 1 |
| | 80 | 78 | 2 |
| | | | 1.2 |
| Oseberg Feltsener, North Sea | 2 | 4 | 2 |
| | 6 | 6 | 0 |
| | 20 | 21 | 1 |
| | 40 | 41 | 1 |
| | 60 | 61 | 1 |
| | 80 | 80 | 0 |
| Average \|Error\|, % of Range | | | 0.8 |

[symbol]

indicates data missing or illegible when filed

TABLE 5
Accuracy Determination, Part 2

| | Concentration, ppm | | \|Error\|, |
|---|---|---|---|
| Oil Source | Actual | Measured | ppm |
| Troll B, North Sea | 2 | 4 | 2 |
| | 6 | 7 | 1 |
| | 20 | 24 | 4 |
| | 40 | 40 | 0 |
| | 60 | 58 | 2 |
| | 80 | 79 | 1 |
| Average \|Error\|, % of Range | | | 1.7 |
| Troll C, North Sea | 2 | 5 | 3 |
| | 6 | 8 | 2 |
| | 20 | 21 | 1 |
| | 40 | 42 | 2 |
| | 60 | 62 | 2 |
| | 80 | 79 | 1 |
| Average \|Error\|, % of Range | | | 1.8 |
| Heavy Crude, Kern River Field, USA | 2 | 2 | 0 |
| | 6 | 6 | 0 |
| | 20 | 20 | 0 |
| | 40 | 40 | 0 |
| | 60 | 61 | 1 |
| | 80 | 80 | 0 |
| Average \|Error\|, % of Range | | | 0.2 |
| Condensate, West Cameron 44, Gulf of | 2 | 7 | 5 |
| Mexico | 6 | 10 | 4 |
| | 20 | 23 | 3 |
| | 40 | 42 | 2 |
| | 60 | 59 | 1 |
| | 80 | 76 | 4 |
| Average \|Error\|, % of Range | | | 3.2 |
| Average \|Error\| over all oils, % of Range | | | 1.5 |

Accuracy was quite good, with average error ranging from 0.2 to 3.2% for all the oils tested. The average error over all types of oils was 1.5%.

Extended Range Measurement

The analytical range of Method 1 was explored with standard solutions of Oseberg C oil in Brine 1. The standards were prepared with oil concentrations from 0 to 10,000 ppm. The TD-500D was calibrated to read 1,000 relative fluorescence units (RFU) on channel A and B with the 10,000 ppm standard and distilled-water blank. All of the standards were then measured on channel A and B. The small-aperture cuvette adaptor was used for all measurements. Results are shown in FIG. 9, in the form of a plot of relative fluorescence versus oil concentration.

For channel A, which excites fluorescence with ultraviolet light, fluorescence increased rapidly with concentration, exhibiting a linear relationship with concentration up to 750 ppm. Between 750 ppm and 1,000 ppm, fluorescence became a non-linear function of oil concentration due to the onset of quenching (the inner filter effect). At concentrations greater than 1,000 ppm, the fluorescence was so intense that it saturated the instrument's light detector. Note that the 0-100 ppm measurements (Table 4) were also made with the same optics (channel A, small-aperture cuvette adaptor). This indicates that the same configuration can be used effectively at the low concentrations found in overboard discharge water, as well as the high concentrations found upstream in the water purification system.

Channel B, which excites fluorescence with visible light, gave measurements that were linear up to 2,000 ppm, and followed a 2nd order polynomial curve all the way to 10,000 ppm.

Field Measurements

Various embodiments have been used in the field to monitor the oil content of produced water from a California steam-flood operation. A diagram of the facility is shown in FIG. 10. Samples were taken in rapid succession from the points indicated on the diagram. Measurements were made with a TD-500 Oil-in-Water Analyzer. The TD-500 is an earlier version of the TD-500D, that only had channel A optics. The calibration standard contained 100 ppm of dispersed oil. Analysis results are shown in Table 6.

TABLE 6
Field Analysis Results

| | OIW | Background | Dispersed Oil |
|---|---|---|---|
| Sample Location | Reading | Reading | Concentration, ppm |
| FWKO Out | >120 | 42 | >78 |
| FWKO Out | >120 | 43 | >77 |
| FWKO Out | >120 | 41 | >79 |
| FWKO Out | >120 | 44 | >76 |
| Surge Tank Out | 62 | 40 | 22 |
| Surge Tank Out | 60 | 41 | 19 |
| Surge Tank Out | 59 | 43 | 16 |
| Surge Tank Out | 64 | 41 | 23 |
| Filter Out | 49 | 42 | 7 |
| Filter Out | 52 | 43 | 10 |
| Filter Out | 48 | 42 | 6 |
| Filter Out | 49 | 41 | 8 |

In this case, the heavy crude oil was so fluorescent that the instrument's detector saturated at OIW readings greater than 120 ppm. As demonstrated above, the linear range could have been extended to higher concentrations with a TD-500D because of the availability of channel B.

As expected, the results indicated that the OIW sample readings and the dispersed oil content of the water samples decreased as the water passed through the system. The repeatability of the dispersed oil measurement was not as good as reported above for the synthetic laboratory samples. Since the field samples were collected from a live water system, the actual dispersed oil content of the samples would naturally be more variable than that of synthetic samples prepared in the laboratory.

Solvent-Free Oil-In-Water Analysis Method for High Salinity Samples

While various embodiments of the solvent-free oil-in-water analysis method described above may be generally independent of salinity value, the embodiments may not be viable at salinity values above 150,000 ppm TDS. High salt concentrations tend to cause the surfactant to exceed the cloud point of the surfactant at normal ambient temperatures. This may result in a surfactant-rich layer that floats on top of the water sample instead of creating a homogeneous microemulsion throughout the sample that is required for fluorescence measurements.

Further, even with salinity values below 150,000 ppm, the surfactant dosage as described above is generally high (about 3 mL per 100 mL sample). This results in a higher cost per sample because of the large amount of surfactant required, with corresponding problems related to storage, transporting the surfactant supply to the analysis site, and the cost of disposal of the samples after analysis. In order to expand the use of the previous embodiments to higher salinity waters, the methods were modified as follows.

In various embodiments, the water sample is first diluted with essentially hydrocarbon-free deionized water. The corresponding reduction in salinity allows the surfactant to establish the optically transparent microemulsion required for effective fluorescence measurements. Experimentation showed that the microemulsion structure could be maintained at surfactant concentrations as low as the CMC of the surfactant. In addition to diluting the surfactant treated water sample, the surfactant-free background sample is diluted the same amount.

To determine the amount of dilution, referred to the dilution factor, the salinity of the water sample is first determined (in various embodiments, the salinity may be estimated rather than actually determined). The dilution factor to reduce the salinity to less than about 150,000 ppm TDS is determined. The water sample and the background sample are diluted by the same dilution factor.

The amount of surfactant to add to the diluted water sample is determined by the CMC of the surfactant. While experimentation has shown that amounts as low as the CMC can be used, in certain instances a greater amount of surfactant may be needed. Thus, the amount of surfactant is calculated as about the CMC multiplied by the dilution factor. Once the water sample is diluted and the proper amount of surfactant is added to the diluted water sample, and the background sample is diluted by the same dilution factor, the method proceeds as described above. Once the oil concentration is determined by the fluorometer, the result is then scaled up by the dilution factor to determine the final value.

The amount of surfactant required for the dilution method is significantly less than that required for the non-dilution method. The non-dilution method, as described above, required 3 mL of 100% active surfactant to be added per 100 mL of the water sample. However, experimentation has shown that the amount of surfactant determined by multiplying the CMC of the surfactant by the dilution factor is generally about 4 μL per 100 mL of the water sample, leading to significant cost reductions based on the amount of surfactant used.

FIG. 11 illustrates a general flow chart of various embodiments of a method 1100 for measuring hydrocarbons in a water sample. At step 1105, a salinity value of the water sample is determined. A dilution factor may then be determined (step 1110) to reduce the salinity value of the water sample to less than 150,000 parts per million (ppm) of total dissolved solids (TDS). The water sample may be diluted by the dilution factor using essentially hydrocarbon-free deionized water at step 1115. An amount of surfactant may then be added at step 1120 to the diluted water sample. The amount of surfactant added may be equal to about the critical micelle concentration of the surfactant multiplied by the dilution factor. At step 1125, the hydrocarbon concentration of the diluted surfactant treated water sample may then be measured.

FIG. 12 is a flowchart of yet another exemplary embodiment of a method 1200 for measuring hydrocarbons in a water sample. While some embodiments may be carried out at least in part manually, other embodiments may be performed utilizing one or more non-transitory computer readable media having computer-executable instructions for performing a method by running a software program on a computer, the computer operating under an operating system, the method comprising instructions from the software program for measuring hydrocarbons in a water sample. The instructions may comprise determining a salinity value of a first water sample (step 1205). A dilution factor may then be determined to reduce the salinity value of the first water sample to less than about 150,000 ppm of total dissolved solids at step 1210. At step 1215, the first water sample may then be diluted by the dilution factor using essentially hydrocarbon-free deionized water. An amount of surfactant may be added at step 1220 to the diluted first water sample. The amount of surfactant added may be equal to about the critical micelle concentration of the surfactant multiplied by the dilution factor. At step 1225, a second water sample may be prepared by filtering a portion of the water sample, then diluting the filtered second water sample by the dilution factor using essentially hydrocarbon-free deionized water (step 1230). The fluorescence of the diluted surfactant treated first water sample and the diluted second water sample may be measured at step 1235. The hydrocarbon concentration of the water sample may be determined at step 1240 based on a difference between the fluorescence measurements of the diluted surfactant treated first water sample and the diluted second water sample.

An exemplary computing system may be used to implement various embodiments of the systems and methods disclosed herein. The computing system may include one or more processors and memory. Main memory stores, in part, instructions and data for execution by a processor to cause the computing system to control the operation of the various elements in the systems described herein to provide the functionality of certain embodiments. Main memory may include a number of memories including a main random access memory (RAM) for storage of instructions and data during program execution and a read only memory (ROM) in which fixed instructions are stored. Main memory may store executable code when in operation. The system further may include a mass storage device, portable storage medium drive(s), output devices, user input devices, a graphics display, and peripheral devices. The components may be connected via a single bus. Alternatively, the components may be connected via multiple buses. The components may be connected through one or more data transport means. Processor unit and main memory may be connected via a local microprocessor bus, and the mass storage device, peripheral device(s), portable storage device, and display system may be connected via one or more input/output (I/O) buses. Mass storage device, which may be implemented with a magnetic disk drive or an optical disk drive, may be a non-volatile storage device for storing data and instructions for use by the processor unit. Mass storage device may store the system software for implementing various embodiments of the disclosed systems and methods for purposes of loading that software into the main memory. Portable storage devices may operate in conjunction with a portable non-volatile storage medium, such as a floppy disk, compact disk or Digital video disc, to input and output data and code to and from the computing system. The system software for implementing various embodiments of the systems and methods disclosed herein may be stored on such a portable medium and input to the computing system via the portable storage device. Input devices may provide a portion of a user interface. Input devices may include an alpha-numeric keypad, such as a keyboard, for inputting alpha-numeric and other information, or a pointing device, such as a mouse, a trackball, stylus, or cursor direction keys. In general, the term input device is intended to include all possible types of devices and ways to input information into the computing system. Additionally, the system may include output devices. Suitable output devices include speakers, printers, network interfaces, and monitors. Display system may include a liquid crystal display (LCD) or other suitable display device.

Display system may receive textual and graphical information, and processes the information for output to the display device. In general, use of the term output device is intended to include all possible types of devices and ways to output information from the computing system to the user or to another machine or computing system. Peripherals may include any type of computer support device to add additional functionality to the computing system. Peripheral device(s) may include a modem or a router or other type of component to provide an interface to a communication network. The communication network may comprise many interconnected computing systems and communication links. The communication links may be wireline links, optical links, wireless links, or any other mechanisms for communication of information. The components contained in the computing system may be those typically found in computing systems that may be suitable for use with embodiments of the systems and methods disclosed herein and are intended to represent a broad category of such computing components that are well known in the art. Thus, the computing system may be a personal computer, hand held computing device, telephone, mobile computing device, workstation, server, minicomputer, mainframe computer, or any other computing device. The computer may also include different bus configurations, networked platforms, multi-processor platforms, etc.

Various operating systems may be used including Unix, Linux, Windows, Macintosh OS, Palm OS, MS-DOS, MINIX, VMS, OS/2, and other suitable operating systems. Due to the ever changing nature of computers and networks, the description of the computing system is intended only as a specific example for purposes of describing embodiments. Many other configurations of the computing system are possible having more or less components.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc. and are also not intended to be limiting. Like terms refer to like elements throughout the description.

As used herein, the terms “having”, “containing”, “including”, “comprising”, and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A method for measuring hydrocarbons in a water sample, comprising:
determining a salinity value of the water sample;
determining a dilution factor to reduce the salinity value of the water sample to less than 150,000 ppm of total dissolved solids;
diluting the water sample by the dilution factor using essentially hydrocarbon-free deionized water;
adding an amount of surfactant to the diluted water sample equal to about the critical micelle concentration of the surfactant multiplied by the dilution factor; and
measuring the hydrocarbon concentration of the diluted surfactant treated water sample.

2. The method of claim 1, wherein the dilution factor ranges from about 1.5 to about 3.

3. The method of claim 1, further comprising scaling up the measured hydrocarbon concentration by the dilution factor.

4. The method of claim 1, further comprising preparing a background sample by filtering a second water sample and diluting the filtered second water sample by the dilution factor using essentially hydrocarbon-free deionized water.

5. The method of claim 4, wherein filtering the second water sample comprises passing the second water sample through a 0.2 micron filter.

6. The method of claim 4, further comprising measuring the hydrocarbon concentration of the filtered second water sample.

7. The method of claim 6, wherein measuring the hydrocarbon concentration of the diluted surfactant treated water sample and the filtered second water sample comprises measuring fluorescence.

8. The method of claim 7, wherein the fluorescence measurements are conducted using ultraviolet light.

9. The method of claim 7, wherein the fluorescence measurements are conducted using visible light.

10. The method of claim 1, further comprising heating the diluted surfactant treated water sample to a cloud point of the surfactant and allowing the water sample to cool until the cloudiness abates prior to measuring the hydrocarbon concentration.

11. The method of claim 1, further comprising filtering the diluted surfactant treated water sample prior to measuring the hydrocarbon concentration.

12. The method of claim 11, wherein filtering the diluted surfactant treated water sample comprises passing the water sample through a 0.2 micron filter.

13. The method of claim 1, wherein the surfactant comprises an aliphatic alcohol ethoxylate.

14. The method of claim 13, wherein the aliphatic alcohol ethoxylate comprises a primary alcohol ethoxylate.

15. The method of claim 13, wherein the aliphatic alcohol ethoxylate comprises a secondary alcohol ethoxylate.

16. The method of claim 1, wherein the water sample comprises produced water from an oil well or natural gas well.

17. A method for measuring hydrocarbons in a water sample, comprising:
determining a salinity value of a first water sample;
determining a dilution factor to reduce the salinity value of the first water sample to less than about 150,000 ppm of total dissolved solids;
diluting the first water sample by the dilution factor using essentially hydrocarbon-free deionized water;
adding an amount of surfactant to the diluted first water sample equal to about the critical micelle concentration of the surfactant multiplied by the dilution factor;
preparing a second water sample by filtering a portion of the water sample;
diluting the filtered second water sample by the dilution factor using essentially hydrocarbon-free deionized water;
measuring the fluorescence of the diluted surfactant treated first water sample and the diluted second water sample; and
determining the hydrocarbon concentration of the water sample based on a difference between the fluorescence measurements of the diluted surfactant treated first water sample and the diluted second water sample.

18. The method of claim 17, wherein the dilution factor ranges from about 1.5 to about 3.

19. The method of claim 17, further comprising scaling up the measured hydrocarbon concentration by the dilution factor.

20. The method of claim 17, wherein filtering the second water sample comprises passing the second water sample through a 0.2 micron filter.

21. The method of claim 17, wherein the fluorescence measurements are conducted using ultraviolet light.

22. The method of claim 17, wherein the fluorescence measurements are conducted using visible light.

23. The method of claim 17, further comprising heating the diluted surfactant treated first water sample to a cloud point of the surfactant and allowing the first water sample to cool until the cloudiness abates prior to measuring the hydrocarbon concentration.

24. The method of claim 17, further comprising filtering the diluted surfactant treated first water sample prior to measuring the fluorescence.

25. The method of claim 17, wherein the surfactant comprises an aliphatic alcohol ethoxylate.

26. The method of claim 25, wherein the aliphatic alcohol ethoxylate comprises a primary alcohol ethoxylate.

27. The method of claim 25, wherein the aliphatic alcohol ethoxylate comprises a secondary alcohol ethoxylate.

28. The method of claim 17, wherein the water sample comprises produced water from an oil well or natural gas well.

29. One or more non-transitory computer-readable media having computer-executable instructions for performing a method by running a software program on a computer, the computer operating under an operating system, the method comprising instructions from the software program for measuring hydrocarbons in a water sample, the instruction comprising:
determining a salinity value of a first water sample;
determining a dilution factor to reduce the salinity value of the first water sample to less than about 150,000 ppm of total dissolved solids;
diluting the first water sample by the dilution factor using essentially hydrocarbon-free deionized water;
adding an amount of surfactant to the diluted first water sample equal to about the critical micelle concentration of the surfactant multiplied by the dilution factor;
preparing a second water sample by filtering a portion of the water sample;
diluting the filtered second water sample by the dilution factor using essentially hydrocarbon-free deionized water;
measuring the fluorescence of the diluted surfactant treated first water sample and the diluted second water sample; and
determining the hydrocarbon concentration of the water sample based on a difference between the fluorescence measurements of the diluted surfactant treated first water sample and the diluted second water sample.

30. The non-transitory computer readable media of claim 29, wherein the dilution factor ranges from about 1.5 to about 3.

31. The non-transitory computer readable media of claim 29, further comprising scaling up the measured hydrocarbon concentration by the dilution factor.

32. The non-transitory computer readable media of claim 29, wherein filtering the second water sample comprises passing the second water sample through a 0.2 micron filter.

33. The non-transitory computer readable media of claim 29, wherein the fluorescence measurements are conducted using ultraviolet light.

34. The non-transitory computer readable media of claim 29, wherein the fluorescence measurements are conducted using visible light.

35. The non-transitory computer readable media of claim 29, further comprising heating the diluted surfactant treated first water sample to a cloud point of the surfactant and allowing the first water sample to cool until the cloudiness abates prior to measuring the hydrocarbon concentration.

36. The non-transitory computer readable media of claim 29, further comprising filtering the diluted surfactant treated first water sample prior to measuring the fluorescence.

37. The non-transitory computer readable media of claim 29, wherein the surfactant comprises an aliphatic alcohol ethoxylate.

38. The non-transitory computer readable media of claim 37, wherein the aliphatic alcohol ethoxylate comprises a primary alcohol ethoxylate.

39. The non-transitory computer readable media of claim 37, wherein the aliphatic alcohol ethoxylate comprises a secondary alcohol ethoxylate.

40. The non-transitory computer readable media of claim 29, wherein the water sample comprises produced water from an oil well or natural gas well.
